(19) United States
(12) Patent Application Publication
(10) Pub. No.: US 2007/0005031 A1
(43) Pub. Date: Jan. 4, 2007

(54) Incontinence collection device

(76) Inventor: Herbert N. Charles, Chatham (CA)

Correspondence Address: CARLSON, GASKEY & OLDS, P.C., 400 WEST MAPLE ROAD, SUITE 350, BIRMINGHAM, MI, 48009, US

(21) Appl. No.: 11/430,457

(22) Filed: May 9, 2006

Related U.S. Application Data
(60) Provisional application No. 60/693,022, filed on Jun. 22, 2005.

(51) Int. Cl. A61M 1/00

(52) U.S. Cl. 604/327

(57) ABSTRACT

An incontinence collection support assembly includes a brief with a pocket for supporting a collection chamber. An outlet tube extends from the collection chamber and includes an outlet valve for selectively draining the contents of the collection chamber. The briefs are fabricated from an elastic material to fit snugly to a wearer. The collection chamber is supported within the pocket and away from the skin of the wearer. The brief fits under an outer garment to effectively conceal the collection chamber. The incontinence collection device is relatively comfortable to wear, concealable under regular outer garments, including shorts, and is easily emptied without removal. Further, the briefs are fabricated with the pocket that isolates and insulates the wearer from contact with the collection chamber to provide an increased level of comfort.

### CROSS REFERENCE TO RELATED APPLICATION

The application claims priority to U.S. Provisional Application No. 60/693,022 which was filed on Jun. 22, 2005.

### BACKGROUND OF THE INVENTION

This invention relates generally to an assembly for collecting and disposing of fluid waste that cannot be retained by the body and to a device for holding a collection chamber in a comfortable position on a wearer.

Incontinent men utilize a device for absorbing and collecting liquid waste that cannot be held within the body due to medical or physical conditions. Conventional methods of absorbing liquid waste include the use of absorbent devices much like diapers that are worn under outer garments that are disposed after becoming saturated with liquid waste.

Another type of device utilized by incontinent men is a collection chamber device. Collection chamber devices accumulate liquid waste for disposal at a later time. The collection chamber is positioned to allow gravity to draw the liquid waste into the collection chamber. For this reason, the specific location of the collection chamber is limited to regions below the pelvis and below a catheter worn by the wearer.

Securement of the collection chamber device in the proper location on the wearer is typically accomplished by utilizing many straps. Many such devices are currently known and include a collection pouch that is strapped to a leg of the wearer to provide for sufficient gravitational forces to evacuate the liquid waste into the collection chamber. As appreciated, the wearing of a collection chamber around ones leg is limiting and uncomfortable. Further, wearing a collection chamber on ones leg limits activity and prevents the wearer from wearing shorts or many other types of garments.

There is also known a device that includes a collection chamber worn around the abdomen region of the wearer that depends on pressure generated by the wearer in evacuating liquid waste to draw the liquid waste from the crotch region upward to the collection chamber secured about the abdomen of the wearer. This device does not provide the comfort and mobility desired by persons with this affliction. Further, because this device depends on pressures created by the wearer it may not be feasible for all persons with incontinent conditions. Certain incontinent conditions prevent the wearer from producing enough internal pressure to drive liquid waste upward from the crotch region into a collection chamber secured about the abdomen of the wearer.

Accordingly, there is a need for a device to accumulate liquid waste that is comfortable and wearable to allow incontinent males to participate in activities while comfortably and dependably accumulating liquid waste for later disposal.

### SUMMARY OF THE INVENTION

An example incontinence collection support assembly includes a brief with a pocket for supporting a collection chamber. An outlet tube extends from the collection chamber and includes an outlet valve for selectively draining the contents of the collection chamber.

The briefs are fabricated from an elastic material to fit snugly to a wearer. The briefs are of boxer brief configuration to provide the desired support and concealment of the collection chamber. The pocket is closed on three sides and opens at a top portion to provide for upward extension of an outlet tube from the pocket. The pocket is also constructed to provide an opening inward toward the wearer. The collection chamber is supported within the pocket and away from the skin of the wearer. The brief fits under an outer garment to effectively conceal the collection chamber. The pocket is disposed in the crotch region of the wearer to provide the comfort and desired concealment of the collection chamber. Further, the pocket provides an opening through which an inlet tube extends for attachment to a catheter worn by the user. The pocket is configured such that the material comprising the collection chamber is not in contact with the wearer's skin. This provides a level of comfort not provided in prior art devices.

Accordingly, the example assembly provides an incontinence collection device that is relatively comfortable to wear, concealable under regular outer garments, including shorts, and is easily emptied without removal. Further, the briefs are fabricated with the pocket that isolates and insulates the wearer from contact with the collection chamber to provide an increased level of comfort.

These and other features of the present invention can be best understood from the following specification and drawings, the following of which is a brief description.

### BRIEF DESCRIPTION OF THE DRAWINGS

FIG. 1 is a schematic view of an example brief for supporting an incontinence collection device.

FIG. 2 is a cross-section of the example brief and incontinence collection device.

FIG. 3 is another cross-section of the example brief and incontinence collection device.

FIG. 4 is a front view of an example collection chamber.

FIG. 5 is a side view of the example collection chamber.

### DETAILED DESCRIPTION OF THE PREFERRED EMBODIMENT

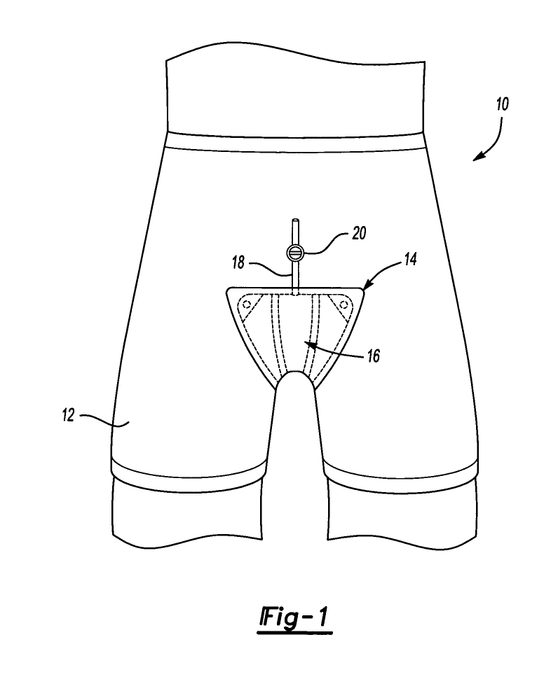

Referring to FIG. 1, an example incontinence collection support assembly 10 includes a brief 12 including a pocket 14. The pocket 14 defines a support cavity for a collection chamber 16. An outlet tube 18 extends from the collection chamber 16 and includes an outlet valve 20 for selectively draining the contents of the collection chamber 16.

The briefs 12 are fabricated from an elastic material to fit snugly to a wearer. The briefs 12 are of boxer brief configuration to provide the desired support and concealment of the collection chamber 16. The boxer brief 12 includes a portion that envelops an upper portion of the wearer's leg while snugly fitting the wearer about the leg and in a crotch region.

The collection chamber 16 is disposed within the pocket 14. The pocket 14 is closed on three sides and opens at a top portion 17 to provide for the outlet tube 18 to extend upwardly out of the pocket 14. The pocket 14 is also constructed to provide an opening inwardly toward the wearer.

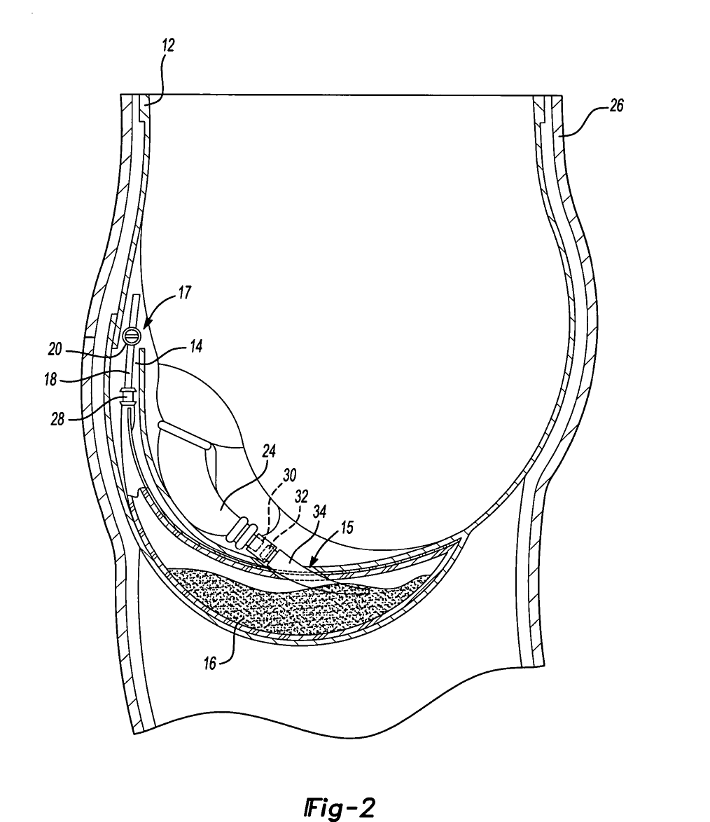

Referring to FIG. 2, the collection chamber 16 is supported within the pocket 14 and away from the skin of the wearer. The pocket 14 includes material on either side of the collection chamber 16 such that a portion of the pocket 14 is disposed between the wearer and the collection chamber 16.

The brief 12 fits under an outer garment 26 to effectively conceal the collection chamber 16. The pocket 14 is disposed in the crotch region of the wearer to provide comfort and conceal the collection chamber 16. Further, the pocket 14 provides an opening 15 through which an inlet tube 22 extends for attachment to a catheter 24 worn by the user. The pocket 14 is configured such that the material comprising the collection chamber 16 is not in contact with the wearer's skin. This provides a level of comfort not provided in prior art devices.

The collection chamber 16 includes an outlet coupling 28 to which the outlet tube 18 is attached. The collection chamber 16 also includes an inlet coupling 32 to which the inlet tube 34 is attached. A backflow prevention valve 30 is disposed within or adjacent the inlet coupling 32 to prevent outflow of the contents of the chamber 16.

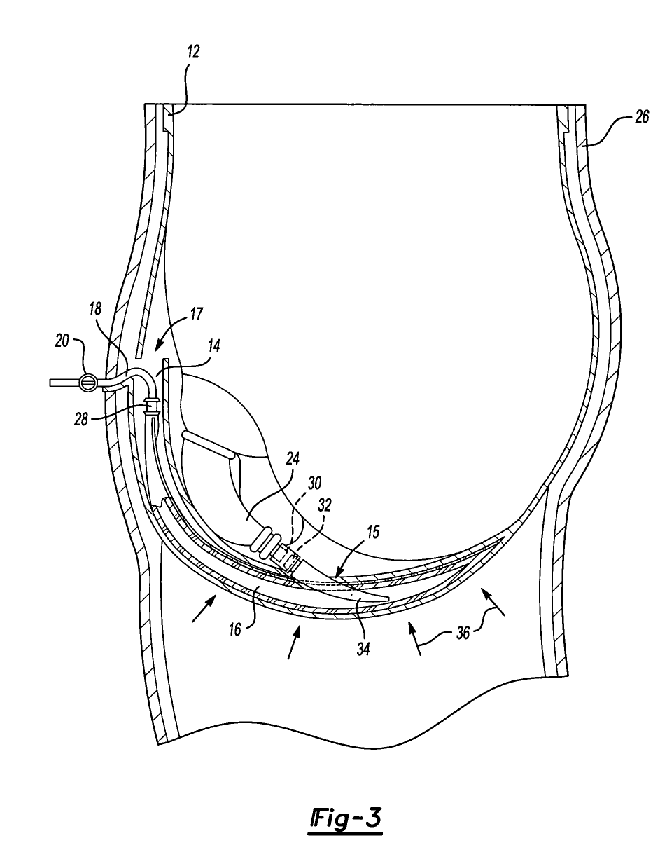

Referring to FIG. 3, the assembly 10 provide for the easy emptying of the contents of the collection chamber 16 without the need to entirely remove the outer garment 26. Emptying of the collection chamber 16 is accomplished by applying pressure to the collection chamber as indicated by arrows 36. The pressure can be applied through the outer garment 26 without revealing or exposing the collection chamber 16. The extended outlet tube 18 is then directed into a suitable receptacle and the outlet valve 20 opened. With the outlet valve opened, pressure applied as indicated at 36 exhausts fluids from the collection chamber 16. The desecrate emptying of the collection chamber 16 provided by the briefs 12 eliminates the need to remove the collection chamber 16 and thereby provides an increased amount of freedom to a wearer afflicted with incontinence.

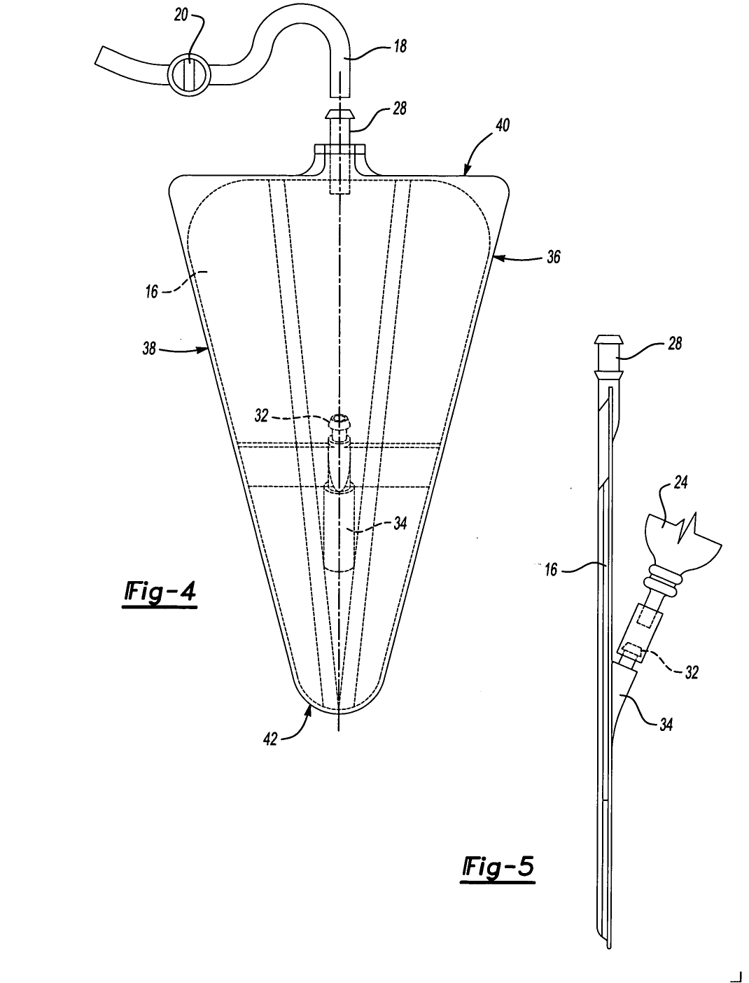

Referring to FIGS. 4 and 5, the example collection chamber 16 is configured for wearing in the crotch region of a user. The collection chamber includes a top 40 and sides 36, 38 that taper from distal ends of the top 40 to a bottom surface 42 that is much shorter than the top 40 to provide a generally triangular shape. The collection chamber 16 includes the inlet tube 34 disposed below the outlet tube 18. The inlet tube 34 being disposed below the outlet provides for the use of gravity to channel waste liquid from the wearer into the collection chamber 16.

As appreciated, some wearers that experience incontinence do not posses the musculature required to provide sufficient force to expel fluid upwardly. The example collection device aids such afflicted wearers by aiding in the drainage of fluids by including the inlet in a substantially downward direction.

The example collection chamber 16 is fabricated with a plastic material that is bonded about an external edge to provide the sealed collection chamber 16. The bonding of the plastic material is accomplished utilizing known adhesive and attachment means and technologies. The specific material utilized for the constructing the collection chamber 16 is plastic material that is substantially impervious to degradation caused by waste fluids. Further, the material is selected so as to provide a comfortable surface texture and composition that does not negatively affect sensitive skin areas of the user.

Accordingly, the example assembly 10 provides an incontinence collection device that is relatively comfortable to wear, concealable under regular outer garments, including shorts, and is easily emptied without removal. Further, the briefs 12 are fabricated with the pocket 14 that isolates and insulates the wearer from contact with the collection chamber 16 to provide an increased level of comfort.

Although a preferred embodiment of this invention has been disclosed, a worker of ordinary skill in this art would recognize that certain modifications would come within the scope of this invention. For that reason, the following claims should be studied to determine the true scope and content of this invention.

What is claimed is:

1. An incontinence collection assembly comprising:
a garment adapted to be worn by a user including a pocket adapted for supporting a collection chamber in a crotch region of the user; the collection chamber including an outlet tube disposed above an inlet tube, said outlet tube including an outlet valve selectively movable between an open and closed position; and
a catheter worn by the user and in fluid communication with a first end of the inlet tube, wherein fluid is communicated from the first end of the inlet tube to a second end of the inlet tube attached to the collection chamber at a point below the catheter such that fluid flows from the catheter through the inlet tube to the collection chamber.

2. The assembly as recited in claim 1, wherein the pocket includes an open top portion for receiving the collection chamber and an inlet slot for receiving the catheter.

3. The assembly as recited in claim 2, wherein the inlet slot is disposed at a lower portion of the pocket.

4. The assembly as recited in claim 1, wherein the garment comprises an undergarment adapted to be worn under outer clothes of the user.

5. The assembly as recited in claim 4, wherein the garment comprises an elastic material for supporting the collection chamber in the crotch region of the user.

6. The assembly as recited in claim 1, wherein the collection chamber includes a back flow prevention valve disposed between the second end of the inlet tube and the collection chamber.

7. The assembly as recited in claim 1, wherein the collection chamber comprises a triangular bag having a top segment, first and second side segments that converge downwardly from opposite ends of the top segment, and a bottom segment.

8. The assembly as recited in claim 1, wherein the outlet tube comprises a rigid tube extending from the collection chamber and a flexible tube extending from the rigid tube, where the flexible tube includes the outlet valve.

9. The assembly as recited in claim 1, wherein fluid is exhausted from the collection chamber by application of pressure on the collection chamber while supported within the garment.

10. A concealed incontinence collection device comprising:
an undergarment comprised of an elastic material and including a pocket adapted for supporting a collection chamber in a crotch region of a wearer; said collection chamber comprising a first sheet of material attached to a second sheet of material at an outer periphery;
an inlet tube including a first end attached to the collection chamber to communicate fluid from a catheter worn by the user; and
an outlet tube attached to the collection chamber at a point above the inlet tube such that fluid drains from the catheter through the inlet tube into the collection chamber.

11. The device as recited in claim 10, wherein the pocket comprises a rectangular sheet of elastic fabric attached to the undergarment on three sides.

12. The device as recited in claim 10, wherein the garment includes a slot disposed within the pocket providing access to the pocket by a user wearing the undergarment.

13. The device as recited in claim 10, including a back flow prevention valve for preventing the flow of fluid out of the collection chamber out the inlet tube.

14. The device as recited in claim 10, wherein fluid is exhausted from the collection chamber through the outlet tube by application of pressure on the collection chamber.

15. The device as recited in claim 10, wherein the collection chamber is substantially triangular including a top segment, a first and second segment extending from opposite ends of the top segment and converging at a bottom segment, where the bottom segment is smaller than the top segment.

16. The device as recited in claim 10, wherein the catheter comprises a condom catheter.
